(19) United States
(12) Patent Application Publication
(10) Pub. No.: US 2020/0276256 A1
(43) Pub. Date: Sep. 3, 2020

(54) SKIN CARE COMPOSITION

(71) Applicant: ISN Pharma SP.Z.O.O., Warszawa, Woj. Mazowieckie (PL)

(72) Inventor: Sylwia Nowak, Warszawa (PL)

(21) Appl. No.: 16/609,096

(22) PCT Filed: Apr. 27, 2018
(86) PCT No.: PCT/EP2018/060875

§ 371 Date: Oct. 28, 2019

(30) Foreign Application Priority Data
Apr. 28, 2017 (PL) P421457.2

(51) Int. Cl. A61K 36/41; A61K 9/06; A61K 9/00; A61K 47/44; A61K 47/10; A61K 47/22; A61P 31/22

(52) U.S. Cl. CPC A61K 36/41; A61K 9/06; A61K 9/0053; A61P 31/22; A61K 47/10; A61K 47/22; A61K 47/44

(57) ABSTRACT

The present invention relates to skin care compositions and methods of making said compositions, particularly topical compositions, comprising extract or juice from the plant Kalanchoe pinnata. The invention further concerns topical compositions comprising extracts from Kalanchoe pinnata and Kalanchoe daigremontiana plants for administration to human skin. The compositions are therapeutically effective for hydrating and conditioning the skin to improve its condition and are additionally shown to be effective at treating some dermal conditions or skin disorders, in particular, psoriasis, atopic dermatitis, allergic contact dermatitis, acne and herpes virus infection.

### RELATED APPLICATIONS

This application is a national phase application filed under 35 USC § 371 of PCT Application No. PCT/EP2018/060875 with an International filing date of Apr. 27, 2018, which claims priority of PL Patent Application P421457.2 filed Apr. 28, 2017. Each of these applications is herein incorporated by reference in its entirety for all purposes.

### FIELD OF THE INVENTION

This invention relates to a skin care composition comprising extract, for example the juice, from Kalanchoe pinnata or Kalanchoe daigremontiana plant for topical administration to human skin and further to a composition comprising extracts from both Kalanchoe pinnata and Kalanchoe daigremontiana plants. In particular, the invention relates to a method of making such compositions and to the uses, particularly cosmetic and medical uses, thereof.

### BACKGROUND

In the field of skin care, a wide variety of products have been developed and commercialised for enhancing skin condition.

Such products are typically formulated as gels, creams, rinses, film-forming compositions, transdermal patches, sprays, or pastes aimed at providing easy application to the skin and effective absorption of components in the formulation. Such formulations must also be stable for reasonable storage periods.

However, the art is continually seeking improved solutions wherein a skin care product both enhances skin condition and is particularly effective in the treatment of one or more common medical complaints associated with the skin.

For example, compositions formulated to combat common adverse dermal disorders such as dermatitis, acne, rosacea, psoriasis, atopic dermatitis and/or allergic contact dermatitis. Skin products directed at alleviating dermal disorders may therefore also frequently include a therapeutically active agent, such as a naturally derived plant extracts, such as aloe vera, or a manufactured ingredient or chemical, such as benzoyl peroxide or hexachlorophene. Other more severe skin complaints, such as those induced by viral infection may also be treated with topical formulations and one or more active ingredient including an antiviral.

In the case of some particular dermal complaints, such as psoriasis and atopic dermatitis, the routinely used treatment prescribed by dermatologists includes application of a formulation comprising a glycocorticosteroid. Steroids are prescription drugs and require frequent application of 1 to 3 times per day for at least 2 weeks. However, steroids are undesirable for a number of reasons: they are expensive for the patient and/or health services and often lead to serious side effects which include inter alia: systemic hormonal changes, skin atrophy, eczema, steroid acne, erythema, vascular purpura, telangiectasia, skin irritation, e.g. burning sensation, reddening, extreme dryness and sometimes bacterial or mycotic superinfections. Moreover, the curative effects of steroids are short-term and after the end of the treatment period the lesions and other physical symptoms associated with psoriasis and atopic dermatitis can re-appear in an aggravated form.

Other treatment options for dermal conditions such as psoriasis include compositions comprising urea, salicylic acid and/or lactic acid. However, these treatments fail to provide a sufficient long term solution for patients; they reduce the superficial appearance of squama on the surface of psoriatic plaque but do not prevent or alleviate regenerating lesions or allow the skin to heal fully. Further, by using such preparations, skin cannot be exposed to sunlight.

Although, medicinal plants are commonly applied in the field of topical formulations, there is a continuing need to identify extracts and develop useful formulations of those extracts to yield solutions for both skin enhancement and clinical treatment of common dermatological conditions. This is particularly true of skin psoriasis, which affects from 1 to 3% of the population.

The treatment of viral skin infections with topical anti-viral medication remains challenging. Where specific viral skin conditions are concerned, a common and problematic pathogen is the Human herpesvirus 1 (HHV-1, herpes simplex virus type 1, HSV-1) which belongs to alpha herpesvirus subfamily, Herpesviridae family. A characteristic feature of this virus is the ability to establish latency in neuronal nuclei, such that HHV-1 is never eliminated from the body and reactivates. A continuing problem with antiviral drugs for treating dermal herpes is that they may only alleviate clinical symptoms by suppressing replication but do not eliminate the virus and thus it frequently re-occurs.

Polish patent application no. PL 398082 discloses a composition which contains from 36% to 52% w/w of phyto product in the form of juice or leaf pulp of two Kalanchoe plant species to improve animal health and condition. For example: poultry resistance to pathogens is greater and the susceptibility to pathogens (micro-organisms) is reduced. However, there is no indication that such extracts from either of these plants, alone or in combination would be useful in the health of humans, or be useful in the treatment of a particular human condition such as dermatitis or herpes

The applicant has recognised a continuing need to identify and develop a product which enhances skin condition and provides a clinically effective treatment for specific dermal complaints, such as those described herein. It is from this need that the present invention has arisen.

### SUMMARY OF INVENTION

The present invention relates to skin care compositions comprising the extract from Kalanchoe pinnata or Kalanchoe daigremontiana. The present invention additionally relates to skin care compositions comprising extracts from both Kalanchoe pinnata and Kalanchoe daigremontiana. The extract(s) may include or comprise the juice of from Kalanchoe pinnata or Kalanchoe daigremontiana plants.

The compositions disclosed herein have been shown to be therapeutically effective for skin conditioning and combating specific medical skin disorders. The invention therefore particularly concerns a composition for topical administration comprising either a therapeutically effective amount of Kalanchoe pinnata extract, or a therapeutically effective amount of Kalanchoe daigremontiana extract, or a combination of the two extracts together. The extracts may be derived from the juice of the pulp of the plant and/or from the juice derived from the leaves and/or the stalk. In embodiments, the composition is formulated with at least one cosmetically or pharmaceutically acceptable excipient to provide a cosmetically suitable topical product.

The inventors have shown that such a composition is useful in the regeneration and recovery of the natural protective layer of the skin. It is also shown that the composition is effective in soothing irritation and inflammation of human skin. Such compositions are found to reduce or eliminate roughness, exfoliation, pulling and burning and soothe itching after a single application. The extracts alone or when utilised in combination appear highly effective for treating, re-conditioning and regenerating healthy skin cells. Such compositions therefore present new applications in the area of topical plant-derived therapeutic compositions.

The invention also concerns a new composition comprising a therapeutically effective amount Kalanchoe daigremontiana extract in the range of 0.01% to 30% w/w and at least one cosmetically or pharmaceutically acceptable excipient for treatment to skin. The invention has been shown to be particularly useful for the treatment of disorders of the skin such as a herpes simplex (HHV-1) infection. Kalanchoe daigremontiana extract, has been found by the applicants to have a positive effect in cells infected with the herpes virus type 1 (HHV-1) when compared to a current known treatment in a number of in vitro-cell tests on human keratinocytes. More preferably, the composition in such an embodiment comprises at least 0.1% or 1% Kalanchoe daigremontiana extract.

In some aspects, the compositions of the invention comprise at least 1% w/w of Kalanchoe pinnata extract or Kalanchoe daigremontiana extract but more preferably 1-30% w/w. In other compositions there may comprise 1-30% w/w of either or both Kalanchoe pinnata and Kalanchoe daigremontiana extracts.

In some embodiments, the composition comprises either 5-15% w/w of Kalanchoe pinnata extract or Kalanchoe daigremontiana extract, or 5-15% of the combination of Kalanchoe pinnata and daigremontiana extracts together. In some embodiments, the content of the extract in the composition is approximately 9-10% w/w.

In some embodiments, the composition may be an epicutaneous formulation and/or formulated as a powder, paste, cream, foam, gel, lotion, ointment or shampoo, for ease of application to a preferred part of the body, e.g. the scalp.

In a preferred embodiment, the composition whether comprising either or both extracts is formulated as a cream. Advantageously, such a cream formulation has excellent skin-absorption properties and enhances the dermal effect of the active extract or extracts. In some aspects the cream may comprise one or more additional excipients as provided herein.

In some embodiments, the composition additionally comprises demineralized water, preferably, 35-75% w/w demineralized water and most preferably approximately 35% w/w.

The at least one excipient may comprises glycerol monostearate, preferably 4-20% w/w glycerol monostearate, preferably approximately 7% w/w.

Moreover, the excipient or excipients in a preferred embodiment of any of the above-mentioned compositions of the invention includes a mixture of fats comprising glycerol monostearate and one or more of beeswax, coconut oil, cetyl alcohol, shea butter and Cannabis sativa (Hemp) Seed Oil.

In one embodiment, the composition comprises 6-65% w/w of the mixture of fats.

Preferably, the composition also comprises 4-28% w/w of beeswax and/or 4-40% w/w of coconut oil and/or 2-25% w/w of cetyl alcohol and/or 2-10% w/w of shea butter.

In one embodiment the composition additionally comprises allantoin, preferably 0.25-0.5% w/w allantoin, which may enhance the therapeutic effect of the formulation.

In a further embodiment, the composition further comprises a preservative, such as phenoxyethanol, preferably 0.5 to 1% w/w phenoxyethanol and most preferably 1% w/w. The preservative prolongs the microbiological stability of the finished product allowing the composition to be stored and used for a longer period.

Further, it is noted that such embodiments have minimal risk of allergic reactions as the applicants have formulated this naturally derived-plant product composition in the absence of fragrance or artificial colourings. Usefully, the composition has been found by patients to enhance the softness, smoothness and hydration of the skin and to promote suppleness after only a single application. Furthermore, the composition of the invention has no adverse side effects.

Further, the invention maybe formulated in accordance any of the above preparations for use with or within a transdermal patch. The invention therefore extends to a dermal patch comprising one or more of any of the above described compositions.

The invention further concerns a composition comprising a therapeutically effective amount of Kalanchoe pinnata extract, or a therapeutically effective amount of Kalanchoe pinnata extract in combination with Kalanchoe daigremontiana extract, in accordance with any of the before described compositions, for use in the treatment of a dermal condition, preferably a human dermal condition.

In a preferred embodiment the dermal condition is selected from skin/xerosis, psoriasis, rosacea, ichthyosis, keratosis, keratoderma, dermatitis, pruritus, acne or eczema.

In a preferred embodiment the condition is atopic dermatitis, allergic contact dermatitis and acne, particularly acne lesions on the back and/or torso.

In one embodiment, where the skin relates to the scalp particularly, the dermal condition maybe seborrheic dermatitis or scalp psoriasis.

In another aspect the disclosure concerns a composition comprising a therapeutically effective amount of Kalanchoe daigremontiana extract in accordance with any of the before described compositions, for use in the treatment of acne, particularly facial and neck acne.

In another aspect the disclosure concerns a composition comprising a therapeutically effective amount of Kalanchoe daigremontiana extract and Kalanchoe pinnata extract in accordance with any of the before described compositions, for use in the treatment of acne, particularly back acne.

In the pharmaceutical and cosmetics market, there are no highly effective, natural skin care preparations useful in treating dermal conditions that share overlapping or common symptoms. However, the applicants have found that regular application of example compositions of the invention substantially reduces or eliminates dermatological ailments and symptoms common to dermal conditions leading to the improvement of the well-being and life quality of the patients.

In a further embodiment, the dermal condition is psoriasis, atopic dermatitis and/or allergic contact dermatitis. The compositions are shown to have an excellent therapeutic effect in the treatment of such disorders: effecting a substantial or complete reduction in lesions and co-related physical symptoms associated with such disorders. It is also shown to provide a regenerating effect in the skin of the patients with these conditions. Furthermore, the composition of the invention does not cause or result in serious side effects that are typically associated with therapeutically active compositions currently prescribed for treating such conditions.

In a further embodiment, the invention concerns a composition for use in treating one or more of the above defined conditions, wherein the composition is applied once, or more preferably, twice daily. For intensive treatment the treatment can involve up to 4 applications daily.

In yet a further embodiment, the invention concerns a composition for use in treating one or more of the above-defined conditions, wherein the duration of the treatment is at least a daily application, preferably a twice-daily application, for at least 5 to 7 consecutive days, preferably at least 7 to 14 days, more preferably at least 28 days.

In a further embodiment, the dermal condition is psoriasis and optionally the treatment duration is 4 to 14 weeks, most preferably 5 to 8 weeks.

In a further embodiment, the dermal condition is allergic contact dermatitis and optionally the treatment duration is 7 to 28 days or 56 days.

In a further embodiment, the dermal condition is atopic dermatitis and optionally the treatment duration is 7 days to 28 days or 56 days.

The invention also relates to a method of enhancing skin condition, comprising topically administering to human skin in need thereof, a composition comprising Kalanchoe pinnata or a therapeutically effective amount of Kalanchoe pinnata in combination with Kalanchoe daigremontiana extract or juice, in accordance with any of the before described compositions.

The invention also concerns a method of treating a dermal condition, comprising topically administering to human skin in need thereof, a composition comprising a therapeutically effective amount of Kalanchoe pinnata or a therapeutically effective amount of Kalanchoe pinnata and Kalanchoe daigremontiana extract or juice, in accordance with any of the before described compositions.

The invention further concerns a method of treating psoriasis, atopic dermatitis or allergic contact dermatitis, comprising topically administering to human skin in need thereof, a composition comprising either Kalanchoe pinnata extract or Kalanchoe pinnata extract and Kalanchoe daigremontiana extract together, in accordance with any of the before described compositions.

The invention also concerns a method of treating acne comprising topically administering to human skin in need thereof, a composition comprising a therapeutically effective amount Kalanchoe daigremontiana or a therapeutically effective amount of Kalanchoe pinnata and Kalanchoe daigremontiana extract or juice, in accordance with any of the before described compositions.

According to a further aspect of the present invention, there is provided a method of treating a herpes simplex (HHV-1) infection comprising topically administering to human skin in need thereof, a composition comprising a therapeutically effective amount Kalanchoe daigremontiana, in accordance with any of the before described compositions.

The invention also comprises a process of producing a topical composition comprising: combining demineralized water, optionally with allantoin, with a mixture of fats comprising glycerol monostearate and one or more of beeswax, coconut oil, cetyl alcohol, shea butter and Cannabis sativa (Hemp) Seed Oil together with Kalanchoe pinnata (or in combination with Kalanchoe daigremontiana) extract or juice to obtain a smooth homogenous mixture.

The invention also comprises a method for making a topical composition comprising the steps of: heating a solution of demineralised water, preferably at 30 to 75 degrees C.; heating a mixture of fats comprising one or more of beeswax, coconut oil, cetyl alcohol, shea butter, Cannabis sativa (Hemp) Seed Oil and glycerol monostearate, preferably at 30-75 degrees C., and stirring until smooth; combining the mixture and solution and heating together, preferably at 30-75 degrees C., and stirring until a smooth and homogenous texture is obtained, followed by cooling, preferably to 20-30 degrees C.; and adding a therapeutically effective amount of Kalanchoe pinnata extract, or a therapeutically effective amount of Kalanchoe pinnata and Kalanchoe daigremontiana extract or juice, to the resulting mixture.

In one embodiment, heating the demineralised water is preferably undertaken at 30-75 degrees C.

In a further embodiment, heating the mixture of fats is preferably undertaken at 30-75 degrees C.

In one embodiment, heating the combined mixture of fats and solution together is undertaken at 30-75 degrees C.

In one embodiment, allantoin, preferably 0.25-0.5% w/w, is added to the demineralised water.

In one embodiment, the method comprises a further final step of homogenising the mixture.

Preferably, the composition comprises 1-30% w/w of Kalanchoe pinnata alone (or in combination with Kalanchoe daigremontiana) extract or juice. More preferably the composition comprises 5-15% of the extract or juice, most preferably approximately 9-10% w/w.

In one embodiment, the composition additionally comprises demineralized water, preferably, 35-75% w/w demineralized water and most preferably approximately 35% w/w.

The at least one excipient may comprises glycerol monostearate, preferably 4-20% w/w glycerol monostearate and most preferably approximately 7% w/w.

In one embodiment, the composition comprises 6-65% w/w of the mixture of fats.

The mixture of fats includes glycerol monostearate and one or more of beeswax, coconut oil, cetyl alcohol, shea butter and Cannabis sativa (Hemp) Seed Oil.

Preferably, the composition comprises 4-28% w/w of beeswax and/or 4-40% w/w of coconut oil and/or 2-25% w/w of cetyl alcohol and/or 2-10% w/w of shea butter.

In a further embodiment, the composition further comprises a preservative, such as phenoxyethanol, preferably 0.5 to 1% w/w phenoxyethanol and most preferably 1% w/w.

Preferably, the composition also comprises allantoin.

In a further aspect, the invention relates to a topical composition obtained by any of the above defined methods.

The extract may include or comprise the juice from any part of Kalanchoe pinnata or Kalanchoe daigremontiana plant.

Other aspects, features and embodiments of the invention will be more fully apparent from the ensuing examples and appended claims.

### BRIEF DESCRIPTION

The following figures provide photographic evidence of the results of a clinical study wherein topical cream formulations, according to embodiments of the disclosure, were tested in three trials and further an in vitro study demonstrating antiviral capability in comparison with a known antiviral in skin cells.

Single Active Example: Kalanchoe pinnata

The formulation of the cream used herein below is in accordance with an example of the disclosure as provided in Example 6.

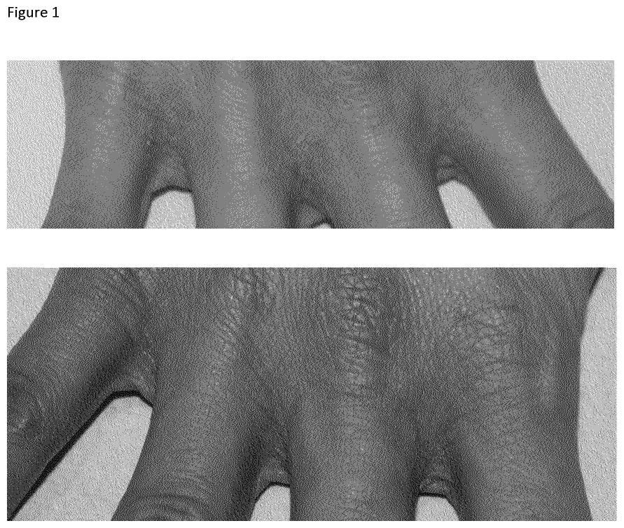

FIG. 1 shows photos of a female suffering atopic dermatitis, before and after treatment with as stated above.

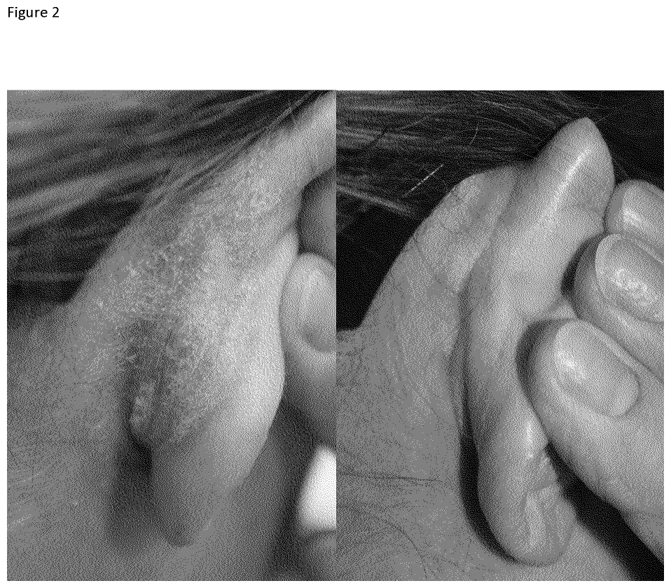

FIG. 2 shows photos of a female suffering psoriasis, before and after treatment with a composition as stated above.

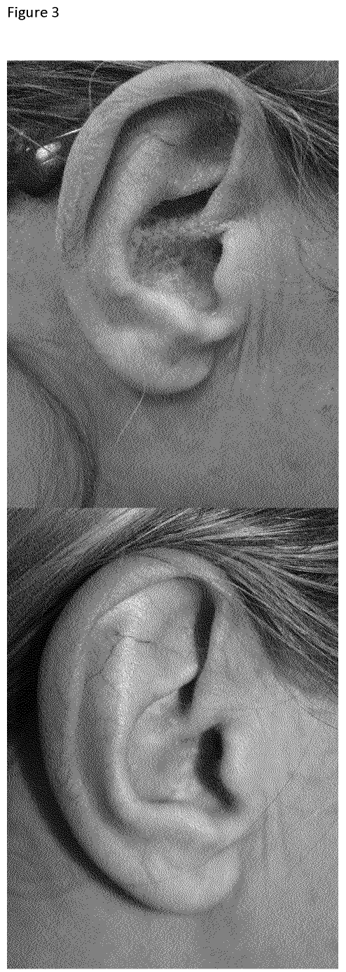

FIG. 3 shows photos of a female suffering psoriasis, before and after treatment with a composition as stated above.

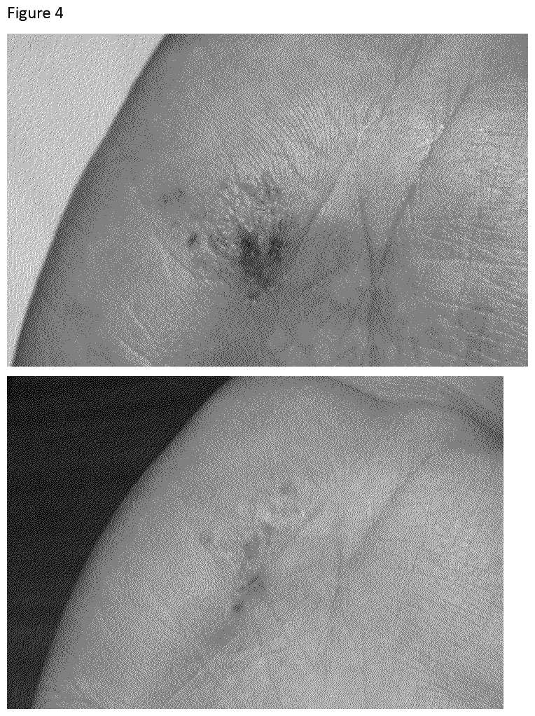

FIG. 4 shows photos of a female suffering allergic contact dermatitis, before and after treatment with a composition as stated above.

### Combination Examples

The formulation of the cream used herein below is in accordance with an example of the disclosure, as provided in Example 6a.

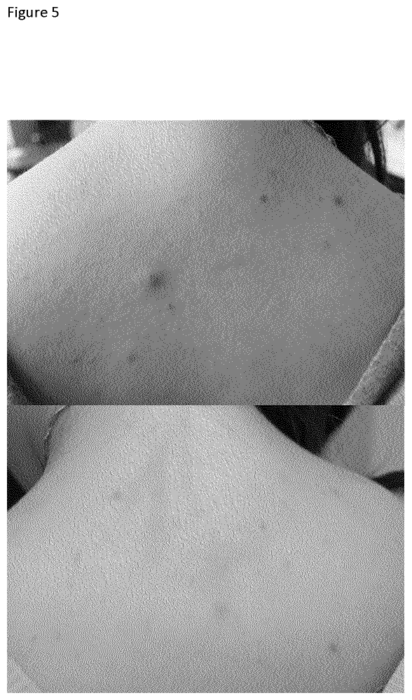

FIG. 5 shows photos of a female suffering back acne, before and after treatment with a composition as stated above.

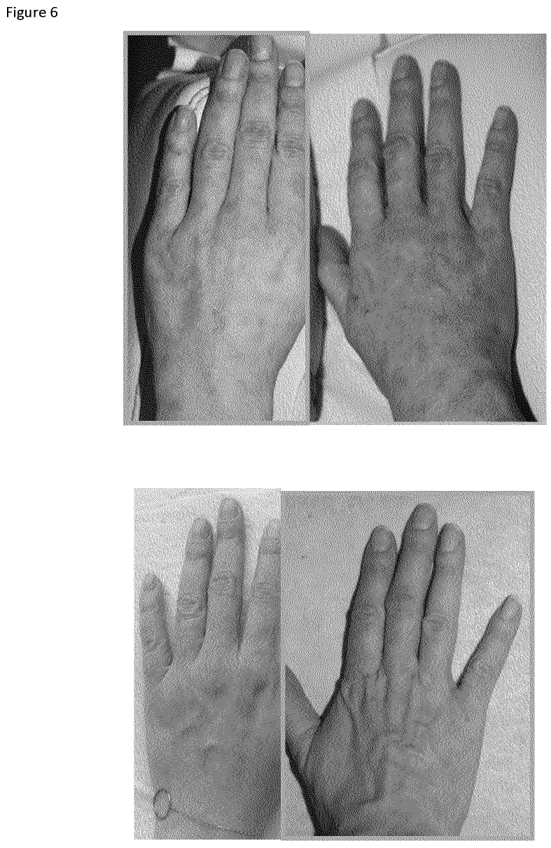

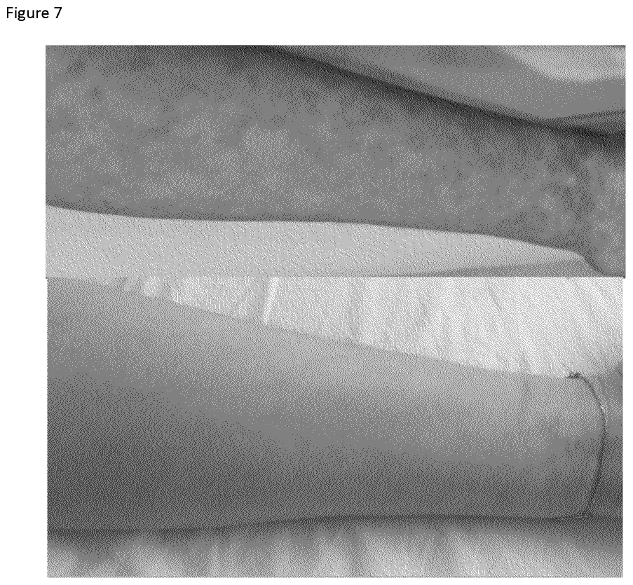

FIGS. 6 and 7 show photos of a female suffering hand and arm psoriasis, before and after treatment with a composition as stated above.

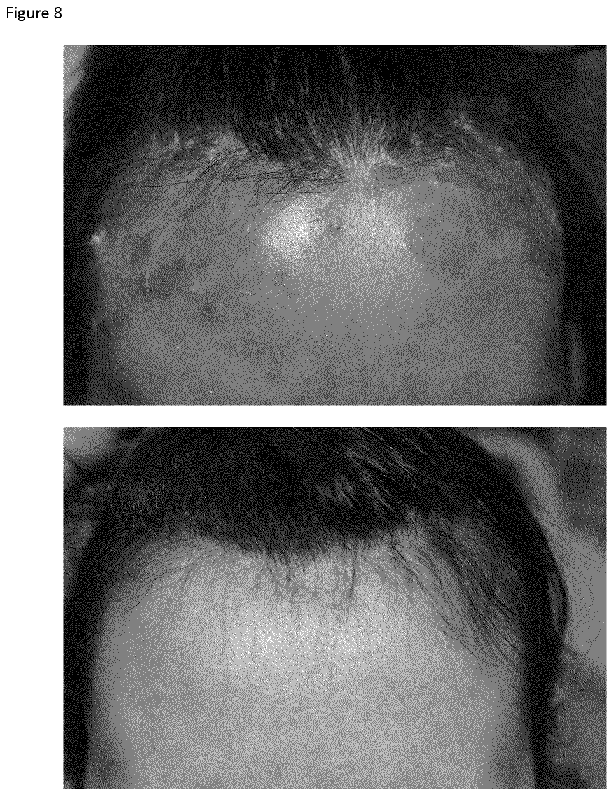

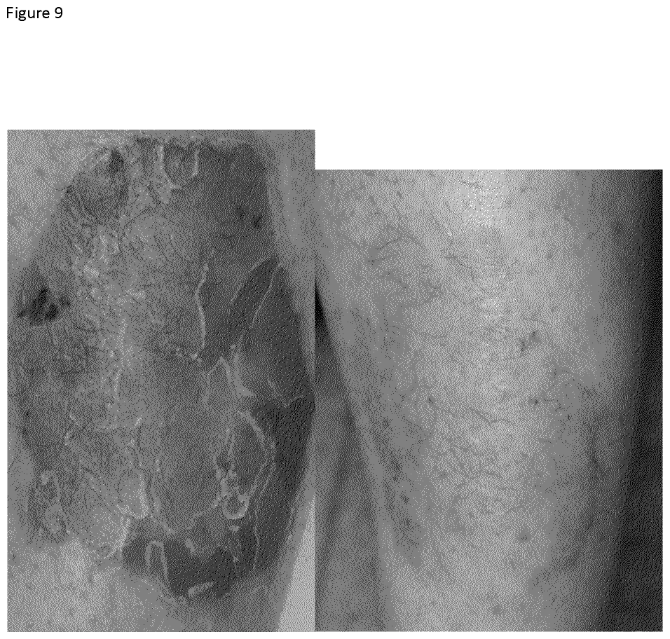

FIGS. 8 and 9 show photos of a female suffering leg and scalp psoriasis, before and after treatment with a composition as stated above.

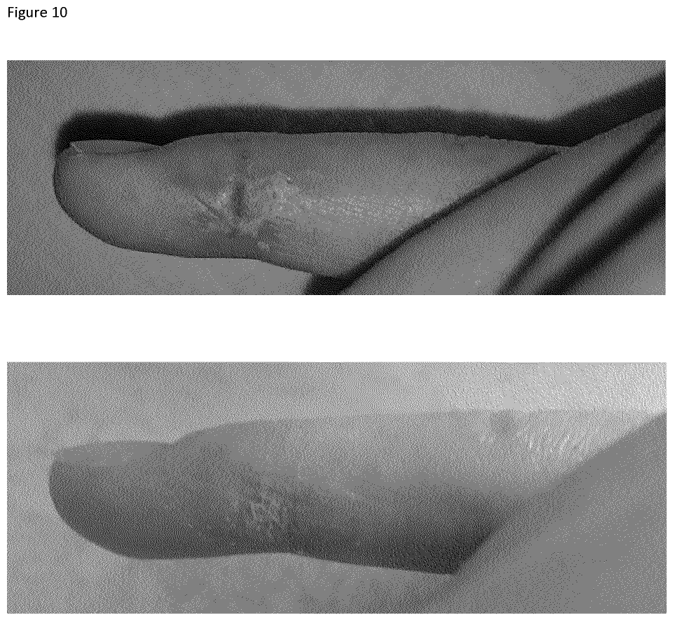

FIG. 10 shows photos of a female suffering allergic contact dermatitis on fingers, before and after treatment with a composition as stated above.

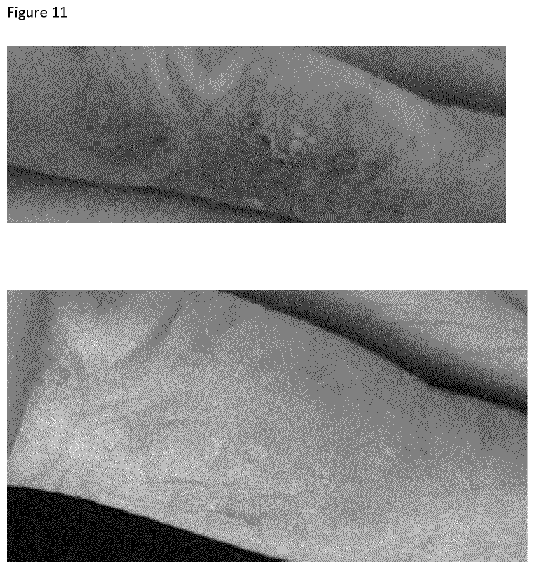

FIG. 11 shows photos of a female suffering atopic contact dermatitis on fingers, before and after treatment with a composition as stated above.

### Single Active Example: Kalanchoe Daigremontiana

An example formulation of the cream may be made in accordance with Example 1b.

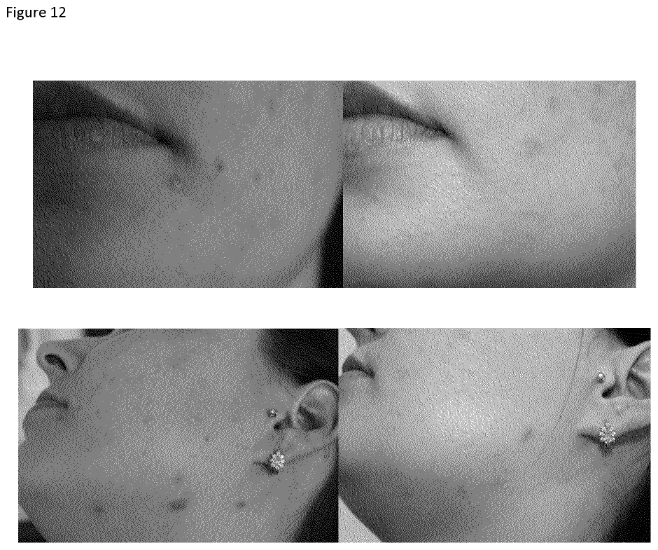

FIG. 12 shows photos of a female suffering from facial acne lesions, before and after treatment with a composition as stated above.

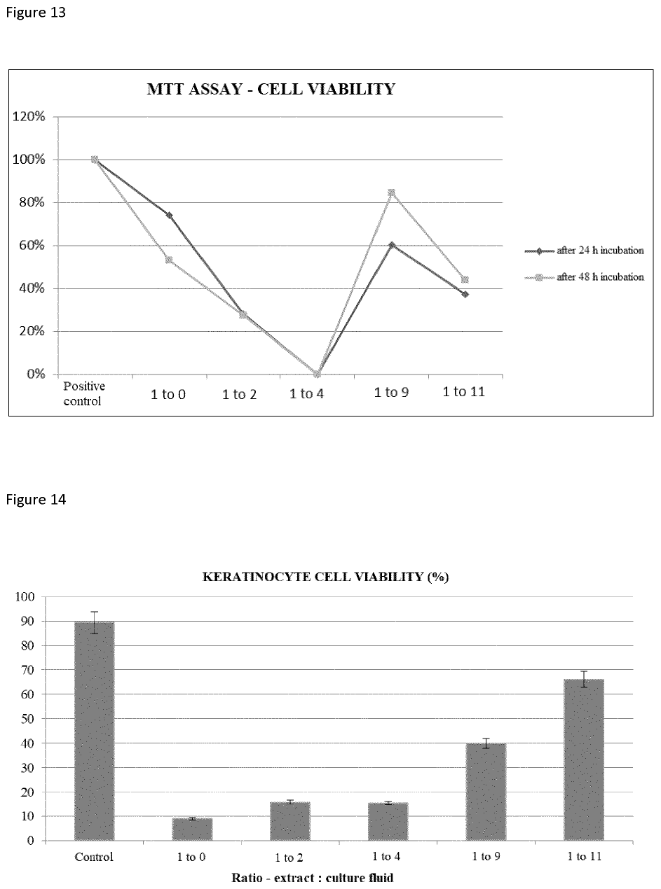

FIG. 13 shows a graphic representation of the cytotoxic effect of Kalanchoe daigremontiana on human keratinocytes.

FIG. 14 shows a graphic representation of human keratinocytes viability in presence of Kalanchoe daigremontiana in varying dilution.

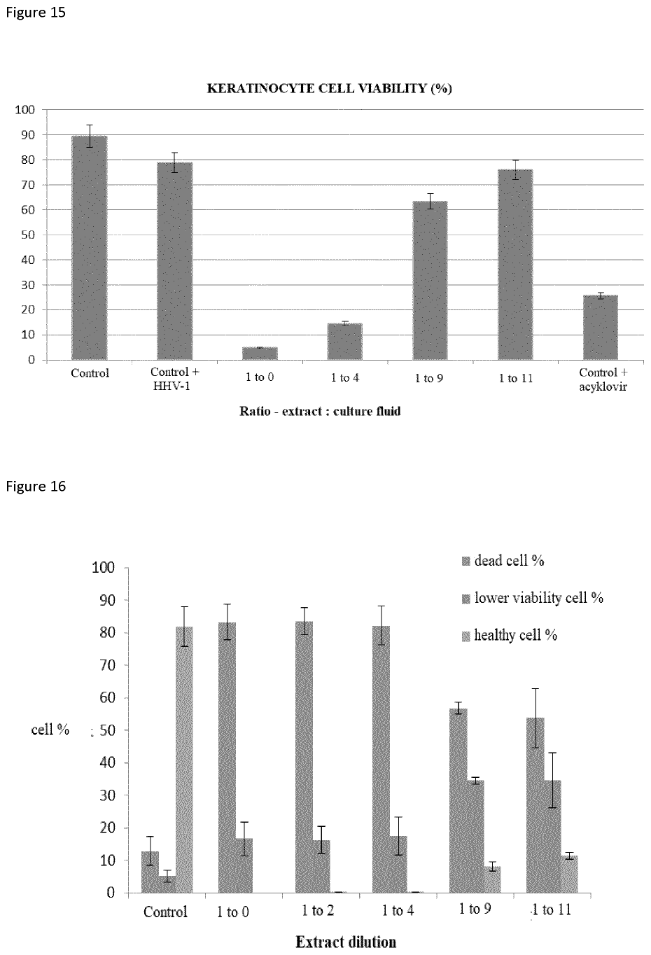

FIG. 15 shows a graphic representation of human keratinocytes viability when Kalanchoe daigremontiana in varying dilution is compared with a known treatment and cells are infected with HHV-1.

FIG. 16 graphically illustrates the percentage estimation of keratinocytes that are dead, of lowered viability or healthy after 24 h incubation with Kalanchoe daigremontiana extract.

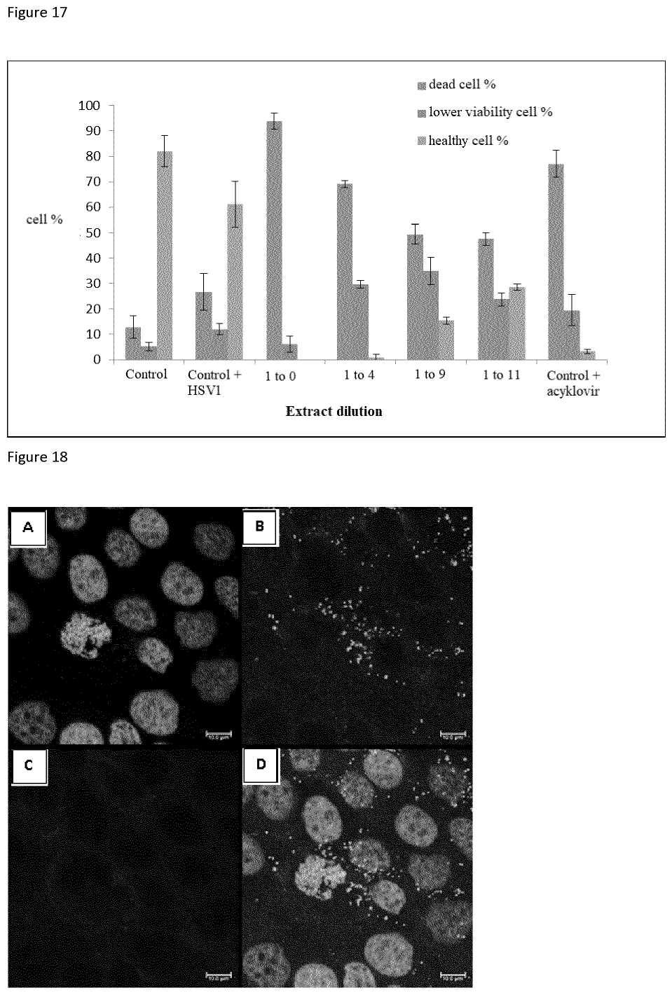

FIG. 17 illustrates the keratinocytes infected with HHV-1 that are dead, of lowered viability or healthy with a control and after 24 h incubation with Kalanchoe daigremontiana extract as compared to acyclovir.

FIG. 18 shows Confocal microscopy images of the cell structure status and positioning 48 hrs after infection with HHV-1.

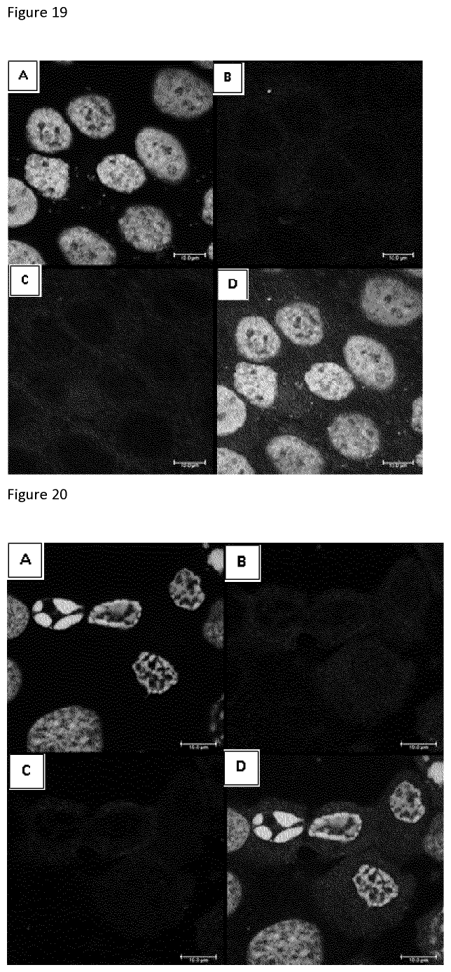

FIG. 19 shows Confocal microscopy images of the cell structure status and positioning 48 hrs after infection with HHV-1 when acyclovir is added to the HaCaT cell line.

FIG. 20 shows Confocal microscopy images of the HaCaT cell line infected with HHV-1 and treated with extract of the invention 1:9 (dilution ratio) at 24 hrs.

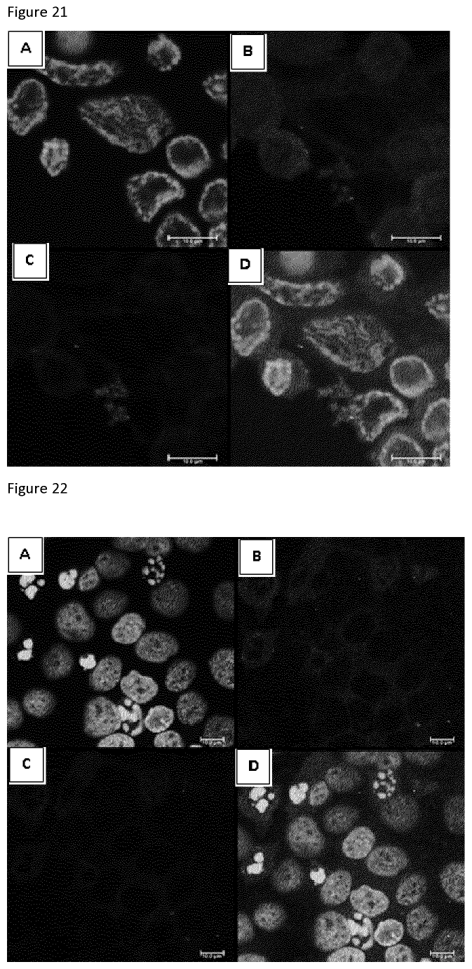

FIG. 21 shows Confocal microscopy images of the HaCaT cell line infected with HHV-1 and treated with extract of the invention 1:9 (dilution ratio) at 48 hrs.

FIG. 22 shows Confocal microscopy images of the HaCaT cell line infected with HHV-1 and treated with extract of the invention 1:11 (dilution ratio) at 24 hrs.

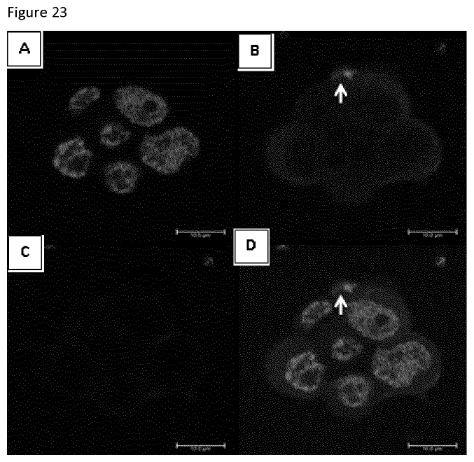

FIG. 23 shows Confocal microscopy images of the HaCaT cell line infected with HHV-1 and treated with extract of the invention 1:11 (dilution ratio) at 48 hrs.

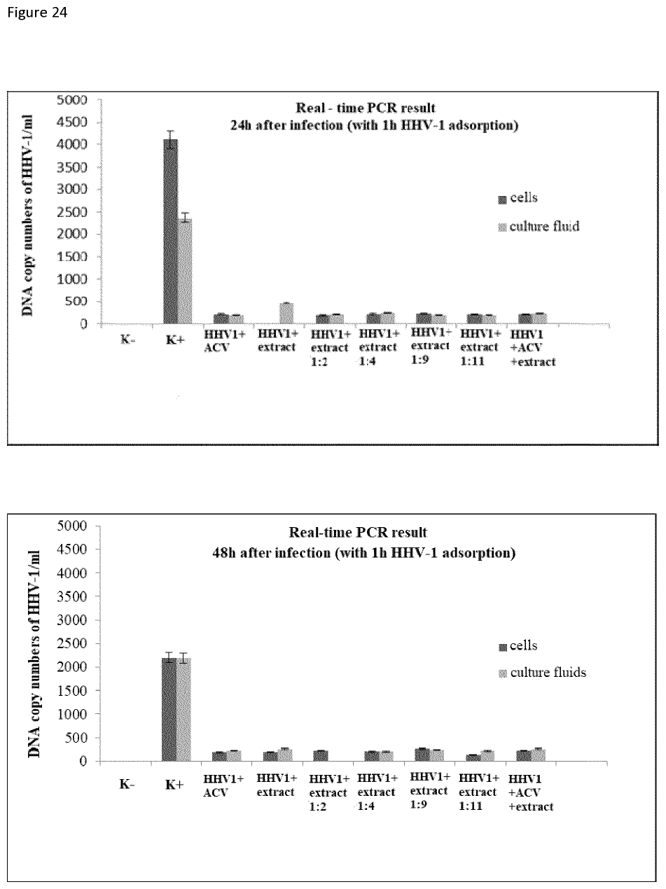

FIG. 24 shows a graphic chart with the results of Real-time PCR analysis of HaCaT cells (at 24 hrs and 48 hrs after infection) with HHV-1 and incubated with Kalanchoe daigremontiana plant extract 1 hr after HHV-1 adsorption.

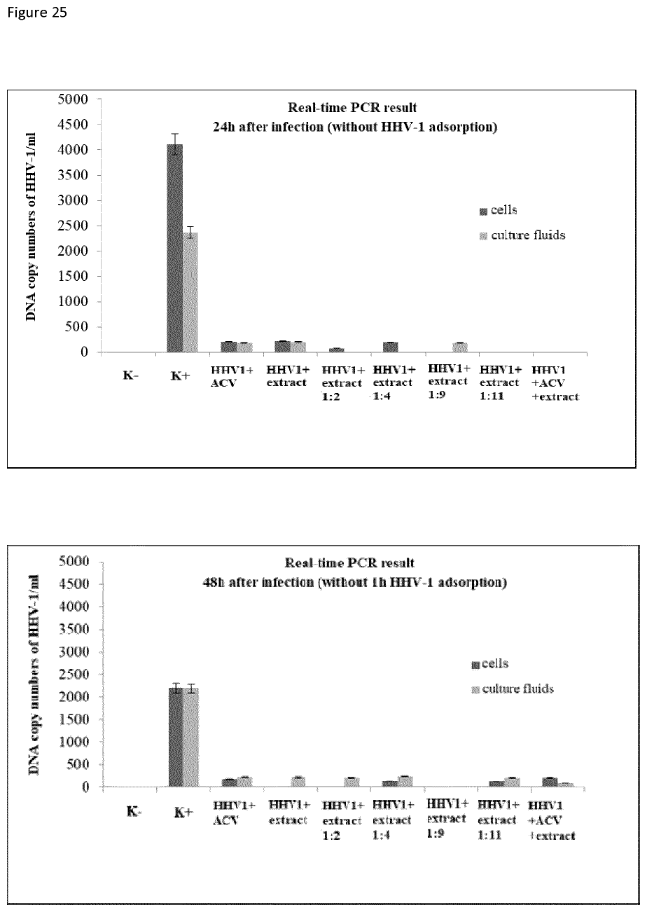

FIG. 25 shows a graphic chart with the results of Real-time PCR analysis of HaCaT cells (at 24 hrs and 48 hrs after infection) with HHV-1 and incubated with Kalanchoe daigremontiana plant extract with no 1 hr pre-adsorption (before culture infection).

### DETAILED DESCRIPTION

The present invention relates to compositions that are topically administered to improve the character of the skin and to combat adverse skin conditions.

Compositions of the invention are usefully employed as skin moisturizers, skin softening agents, skin debridement agents, etc.

In cosmetic formulations, the compositions of the invention may be used with added ingredients that are solely cosmetic. Alternatively, the cosmetic formulation may include ingredients that are both cosmetically efficacious and therapeutically effective, e.g., so-called “cosmeceutical” ingredients.

Compositions of the invention are particularly utilised for treatment clinical dermal conditions and adverse physiological states manifesting dermally, including, without limitation, dry skin/xerosis, psoriasis, ichthyosis, keratosis, keratoderma, dermatitis, pruritus, acne and eczema. Conditions found to be particularly well treated by the compositions of the invention disclosed herein include psoriasis, atopic dermatitis, allergic contact dermatitis and acne.

As used herein, references to compositional ingredients in percent by weight refers to weight percentages (% w/w) based on the total weight of the composition or formulation.

In various embodiments of the invention, the compositions described herein may comprise, consist or consist essentially of the specified ingredients or specific ones thereof. It will be understood that the formulations of the invention may be widely varied, as regards the absolute amounts and relative proportions thereof, in relation to specific examples, and illustrative compositions.

The invention can be illustrated with the following preferred example compositions:

### Examples Comprising Kalanchoe pinnata

### Example 1

1-30% w/w of the extract or juice from Kalanchoe pinnata

4-20% w/w of the glycerol monostearate

4-28% w/w of the beeswax

35-75% w/w of the demineralized water

The extract from Kalanchoe pinnata is an aqueous extract derived from any part of the plant, preferably the leaves and/or the juice from the leaves and/or juice from the plant or other parts of the plant biomass, such as the stalk.

The production process starts with preparation of the fats where glycerol monostearate and beeswax are which is heated up to the temperature of 30-75 degrees C. and stirred until the mixture becomes smooth.

The demineralized water is heated up to 30-75 degrees C.

The fats are then added to the heated demineralized water stirred and homogenised until the mixture is smooth.

The mixture of fats and water is cooled to a temperature of 20-30 degrees C.

The extract of Kalanchoe pinnata is added to the mixture, stirred and homogenised to result in a smooth cream.

### Example 2

1-30% w/w of the extract or juice from Kalanchoe pinnata

4-20% w/w of the glycerol monostearate

4-28% w/w of the beeswax

35-75% w/w of the demineralized water

0.5-1% w/w of phenoxyethanol

The cream production process is completed as per Example 1 with the exception that in the final phase, after the extract of Kalanchoe pinnata is added and stirred, a preservative (phenoxyethanol) is added.

The resulting mixture is then stirred and homogenised.

### Example 3

1-30% w/w of the extract or juice from Kalanchoe pinnata

4-20% w/w of the glycerol monostearate

4-40% w/w of the coconut oil

35-75% w/w of the demineralized water

The cream production process is as provided in Example 1 but using the alternative combination of fats as specified above.

### Example 4

1-30% w/w of the extract or juice from Kalanchoe pinnata

4-20% w/w of the glycerol monostearate

4-40% w/w of the coconut oil

35-75% w/w of the demineralized water

0.5-1% w/w of phenoxyethanol

The cream production process is as provided in Example 1 but using the alternative combination of fats above and in the final phase after the extract of Kalanchoe pinnata is added and stirred, the preservative (phenoxyethanol) is added.

The resulting mixture is then stirred and homogenised.

### Example 5

1-30% w/w of the extract or juice from Kalanchoe pinnata

4-20% w/w of the glycerol monostearate

2-25% w/w of the cetyl alcohol

35-75% w/w of the demineralized water

The cream production process is as provided in Example 3.

### Example 6

1-30% w/w of the extract or juice from Kalanchoe pinnata

4-20% w/w of the glycerol monostearate

2-25% w/w of the cetyl alcohol

35-75% w/w of the demineralized water

0.5-1% w/w of phenoxyethanol

The cream production process is completed as Example 4.

### Example 7

1-30% w/w of the extract or juice from Kalanchoe pinnata

4-20% w/w of the glycerol monostearate

4-40% w/w of the coconut oil

2-10% w/w of the shea butter

35-75% w/w of the demineralized water

The cream production process is as provided in Example 3.

### Example 8

1-30% w/w of the extract or juice from Kalanchoe pinnata

4-20% w/w of the glycerol monostearate

4-40% w/w of the coconut oil

2-10% w/w of the shea butter

35-75% w/w of the demineralized water

0.5-1% w/w of phenoxyethanol

The cream production process is completed as Example 4.

### Example 9

1-30% w/w of the extract or juice from Kalanchoe pinnata

4-20% w/w of the glycerol monostearate

4-40% w/w of the coconut oil

0.25-0.5% w/w of the allantoin

35-75% w/w of the demineralized water

The cream production process is as provided in Example 3, with the exception that allantoin is added to the heated the demineralised water prior to the fats being added thereto.

### Example 10

1-30% w/w of the extract or juice from Kalanchoe pinnata

4-20% w/w of the glycerol monostearate

4-40% w/w of the coconut oil

0.25-0.5% w/w of the allantoin

35-75% w/w of the demineralized water

0.5-1% w/w of phenoxyethanol

The cream production process is as provided in Example 4, with the exception that the allantoin is added to the heated the demineralised water prior to the fats being added thereto.

### Example 11

1-30% w/w of the extract or juice from Kalanchoe pinnata

4-20% w/w of the glycerol monostearate

4-40% w/w of the coconut oil

2-10% w/w of the shea butter

0.25-0.5% w/w of the allantoin

35-75% w/w of the demineralized water

The cream production process is as provided in Example 9.

### Example 12

1-30% w/w of the extract or juice from Kalanchoe pinnata

4-20% w/w of the glycerol monostearate

4-30% w/w of the coconut oil

2-10% w/w of the shea butter

0.25-0.5% w/w of the allantoin

35-75% w/w of the demineralized water

0.5-1% w/w of phenoxyethanol

The cream production process is as provided in Example 10.

### Example 13

1-30% w/w of the extract or juice from Kalanchoe pinnata

4-20% w/w of the glycerol monostearate

4-40% w/w of the Cannabis sativa (Hemp) Seed Oil

2-10% w/w of the shea butter

35-75% w/w of the demineralized water

The cream production process is as provided in Example 3.

### Example 14

1-30% w/w of the extract or juice from Kalanchoe pinnata

4-20% w/w of the glycerol monostearate

4-40% w/w of the Cannabis sativa (Hemp) Seed Oil

2-10% w/w of the shea butter

35-75% w/w of the demineralized water

0.5-1% w/w of phenoxyethanol

The cream production process is as provided in Example 4.

### Example 15

1-30% w/w of the extract or juice from Kalanchoe pinnata

4-20% w/w of the glycerol monostearate

1-5% w/w of the cetyl alcohol

4-40% w/w of the coconut oil

35-75% w/w of the demineralized water

The cream production process is as provided in Example 3.

### Example 16

1-30% w/w of the extract or juice from Kalanchoe pinnata

4-20% w/w of the glycerol monostearate

1-5% w/w of the cetyl alcohol

4-40% w/w of the coconut oil

35-75% w/w of the demineralized water

0.5-1% w/w of phenoxyethanol

The cream production process is as provided in Example 4.

### Example 17

1-30% w/w of the extract or juice from Kalanchoe pinnata

4-20% w/w of the glycerol monostearate

1-5% w/w of the cetyl alcohol

4-40% w/w of the shea butter

35-75% w/w of the demineralized water

The cream production process is as provided in Example 3.

### Example 18

1-30% w/w of the extract or juice from Kalanchoe pinnata

4-20% w/w of the glycerol monostearate

1-5% w/w of the cetyl alcohol

4-40% w/w of the shea butter

35-75% w/w of the demineralized water

0.5-1% w/w of phenoxyethanol

The cream production process is as provided in Example 4.

### Example 19

1-30% w/w of the extract or juice from Kalanchoe pinnata

4-40% w/w of the Cannabis sativa (Hemp) Seed Oil

2-30% w/w of the coconut oil

35-75% w/w of the demineralized water

4-20% w/w of the glycerol monostearate

1-5% w/w of the cetyl alcohol

The cream production process is as provided in Example 3.

### Example 20

1-30% w/w of the extract or juice from Kalanchoe pinnata

4-40% w/w of the Cannabis sativa (Hemp) Seed Oil

2-10% w/w of the coconut oil

35-75% w/w of the demineralized water

4-20% w/w of the glycerol monostearate

1-5% w/w of the cetyl alcohol

0.5-1% w/w of phenoxyethanol

The cream production process is as provided in Example 4.

### Combination Examples Comprising Kalanchoe pinnata and Kalanchoe daigremontiana

### Example 1a

1-30% w/w of the extract or juice from Kalanchoe pinnata

1-30% w/w of the extract or juice from Kalanchoe daigremontiana

4-20% w/w of the glycerol monostearate

4-28% w/w of the beeswax

35-75% w/w of the demineralized water

The extract from Kalanchoe pinnata and extract from Kalanchoe daigremontiana is an aqueous extract derived from any part of the plant, preferably the leaves and/or the juice from the leaves and/or juice from the plant or other parts of the plant biomass, such as the stalk.

The production process starts with preparation of the fats: glycerol monostearate and beeswax which are heated up to the temperature of 30-75 degrees C. and stirred until the mixture becomes smooth.

The demineralized water is heated up to 30-75 degrees C. The fats are then added to the heated demineralized water stirred and homogenised until the mixture is smooth.

The mixture of fats and water is cooled to a temperature of 20-30 degrees C.

The extract of Kalanchoe daigremontiana is added to the mixture, stirred and homogenised to result in a smooth cream.

### Example 2a

1-30% w/w of the extract or juice from Kalanchoe pinnata

1-30% w/w of the extract or juice from Kalanchoe daigremontiana

4-20% w/w of the glycerol monostearate

4-28% w/w of the beeswax

35-75% w/w of the demineralized water

0.5-1% w/w of phenoxyethanol

The cream production process is completed as per Example 1a with the exception that in the final phase, after the extract of Kalanchoe pinnata is added and stirred, the preservative (phenoxyethanol) is added.

The resulting mixture is then stirred and homogenised.

### Example 3a

1-30% w/w of the extract or juice from Kalanchoe pinnata

1-30% w/w of the extract or juice from Kalanchoe daigremontiana

4-20% w/w of the glycerol monostearate

4-40% w/w of the coconut oil

35-75% w/w of the demineralized water

The cream production process is as provided in Example 1a but using the alternative combination of fats as specified above.

### Example 4a

1-30% w/w of the extract or juice from Kalanchoe pinnata

1-30% w/w of the extract or juice from Kalanchoe daigremontiana

4-20% w/w of the glycerol monostearate

4-40% w/w of the coconut oil

35-75% w/w of the demineralized water

0.5-1% w/w of phenoxyethanol

The cream production process is as provided in Example 1a but using the alternative combination of fats above and in the final phase after the extract of Kalanchoe pinnata is added and stirred, a preservative (phenoxyethanol) is added.

The resulting mixture is then stirred and homogenised.

### Example 5a

1-30% w/w of the extract or juice from Kalanchoe pinnata

1-30% w/w of the extract or juice from Kalanchoe daigremontiana

4-20% w/w of the glycerol monostearate

2-25% w/w of the cetyl alcohol

35-75% w/w of the demineralized water

The cream production process is as provided in Example 3a.

### Example 6a

1-30% w/w of the extract or juice from Kalanchoe pinnata

1-30% w/w of the extract or juice from Kalanchoe daigremontiana

4-20% w/w of the glycerol monostearate

2-25% w/w of the cetyl alcohol

35-75% w/w of the demineralized water

0.5-1% w/w of phenoxyethanol

The cream production process is completed as Example 4a.

### Example 7a

1-30% w/w of the extract or juice from Kalanchoe pinnata

1-30% w/w of the extract or juice from Kalanchoe daigremontiana

4-20% w/w of the glycerol monostearate

4-40% w/w of the coconut oil

2-10% w/w of the shea butter

35-75% w/w of the demineralized water

The cream production process is as provided in Example 3a.

### Example 8a

1-30% w/w of the extract or juice from Kalanchoe pinnata

1-30% w/w of the extract or juice from Kalanchoe daigremontiana

4-20% w/w of the glycerol monostearate

4-40% w/w of the coconut oil

2-10% w/w of the shea butter

35-75% w/w of the demineralized water

0.5-1% w/w of phenoxyethanol

The cream production process is completed as Example 4a.

### Example 9a

1-30% w/w of the extract or juice from Kalanchoe pinnata

1-30% w/w of the extract or juice from Kalanchoe daigremontiana

4-20% w/w of the glycerol monostearate

4-40% w/w of the coconut oil

0.25-0.5% w/w of the allantoin

35-75% w/w of the demineralized water

The cream production process is as provided in Example 3a, with the exception that allantoin is added to the heated the demineralised water prior to the fats being added thereto.

### Example 10a

1-30% w/w of the extract or juice from Kalanchoe pinnata

1-30% w/w of the extract or juice from Kalanchoe daigremontiana

4-20% w/w of the glycerol monostearate

4-40% w/w of the coconut oil

0.25-0.5% w/w of the allantoin

35-75% w/w of the demineralized water

0.5-1% w/w of phenoxyethanol

The cream production process is as provided in Example 4a, with the exception that the allantoin is added to the heated the demineralised water prior to the fats being added thereto.

### Example 11a

1-30% w/w of the extract or juice from Kalanchoe pinnata

1-30% w/w of the extract or juice from Kalanchoe daigremontiana

4-20% w/w of the glycerol monostearate

4-40% w/w of the coconut oil

2-10% w/w of the shea butter

0.25-0.5% w/w of the allantoin

35-75% w/w of the demineralized water

The cream production process is as provided in Example 9a.

### Example 12a

1-30% w/w of the extract or juice from Kalanchoe pinnata

1-30% w/w of the extract or juice from Kalanchoe daigremontiana

4-20% w/w of the glycerol monostearate

4-30% w/w of the coconut oil

2-10% w/w of the shea butter

0.25-0.5% w/w of the allantoin

35-75% w/w of the demineralized water

0.5-1% w/w of phenoxyethanol

The cream production process is as provided in Example 10a.

### Example 13a

1-30% w/w of the extract or juice from Kalanchoe pinnata

1-30% w/w of the extract or juice from Kalanchoe daigremontiana

4-20% w/w of the glycerol monostearate

4-40% w/w of the Cannabis sativa (Hemp) Seed Oil

2-10% w/w of the shea butter

35-75% w/w of the demineralized water

The cream production process is as provided in Example 3a.

### Example 14a

1-30% w/w of the extract or juice from Kalanchoe pinnata

1-30% w/w of the extract or juice from Kalanchoe daigremontiana

4-20% w/w of the glycerol monostearate

4-40% w/w of the Cannabis sativa (Hemp) Seed Oil

2-10% w/w of the shea butter

35-75% w/w of the demineralized water

0.5-1% w/w of phenoxyethanol

The cream production process is as provided in Example 4a.

### Example 15a

1-30% w/w of the extract or juice from Kalanchoe pinnata

1-30% w/w of the extract or juice from Kalanchoe daigremontiana

4-20% w/w of the glycerol monostearate

1-5% w/w of the cetyl alcohol

4-40% w/w of the coconut oil

35-75% w/w of the demineralized water

The cream production process is as provided in Example 3a.

### Example 16a

1-30% w/w of the extract or juice from Kalanchoe pinnata

1-30% w/w of the extract or juice from Kalanchoe daigremontiana

4-20% w/w of the glycerol monostearate

1-5% w/w of the cetyl alcohol

4-40% w/w of the coconut oil

35-75% w/w of the demineralized water

0.5-1% w/w of phenoxyethanol

The cream production process is as provided in Example 4a.

### Example 17a

1-30% w/w of the extract or juice from Kalanchoe pinnata

1-30% w/w of the extract or juice from Kalanchoe daigremontiana

4-20% w/w of the glycerol monostearate

1-5% w/w of the cetyl alcohol

4-40% w/w of the shea butter

35-75% w/w of the demineralized water

The cream production process is as provided in Example 3a.

### Example 18a

1-30% w/w of the extract or juice from Kalanchoe pinnata

1-30% w/w of the extract or juice from Kalanchoe daigremontiana

4-20% w/w of the glycerol monostearate

1-5% w/w of the cetyl alcohol

4-40% w/w of the shea butter

35-75% w/w of the demineralized water

0.5-1% w/w of phenoxyethanol

The cream production process is as provided in Example 4a.

### Example 19a

1-30% w/w of the extract or juice from Kalanchoe pinnata

1-30% w/w of the extract or juice from Kalanchoe daigremontiana

4-40% w/w of the Cannabis sativa (Hemp) Seed Oil

2-30% w/w of the coconut oil

35-75% w/w of the demineralized water

4-20% w/w of the glycerol monostearate

1-5% w/w of the cetyl alcohol

The cream production process is as provided in Example 3a.

### Example 20a

1-30% w/w of the extract or juice from Kalanchoe pinnata

1-30% w/w of the extract or juice from Kalanchoe daigremontiana

4-40% w/w of the Cannabis sativa (Hemp) Seed Oil

2-10% w/w of the coconut oil

35-75% w/w of the demineralized water

4-20% w/w of the glycerol monostearate

1-5% w/w of the cetyl alcohol

0.5-1% w/w of phenoxyethanol

The cream production process is as provided in Example 4a.

### Examples Comprising Kalanchoe daigremontiana

### Example 1b

1-30% w/w of the extract or juice from Kalanchoe daigremontiana

4-20% w/w of the glycerol monostearate

4-28% w/w of the beeswax

35-75% w/w of the demineralized water

The extract from Kalanchoe daigremontiana is an aqueous extract derived from any part of the plant, preferably the leaves and/or the juice from the leaves and/or juice from the plant or other parts of the plant biomass, such as the stalk.

The production process starts with preparation of the fats: glycerol monostearate and beeswax which are heated up to the temperature of 30-75 degrees C. and stirred until the mixture becomes smooth.

The demineralized water is heated up to 30-75 degrees C. The fats are then added to the heated demineralized water stirred and homogenised until the mixture is smooth.

The mixture of fats and water is cooled to a temperature of 20-30 degrees C.

The extract of Kalanchoe daigremontiana is added to the mixture, stirred and homogenised to result in a smooth cream.

Results

Patient trials of a cream formulation comprising an extract or juice of Kalanchoe pinnata alone, an extract or juice of daigremontiana (for acne specifically) alone, or a combination of the two extracts were undertaken.

The cream that was tested comprised the formulation of within the ranges specified in the Examples provided herein, particularly as found in Examples: 6, 6a and 1b.

The dosage regimen comprises a twice daily regimen of at least 2 days but most preferably 5, 7 or up to 28 days consecutive application depending on the particular trial.

Patient Compliance

The cream has the form of homogenous emulsion with properly selected consistency, which, in the opinion of the trial participants, enabled them to apply the cream evenly on body parts. The cream consistency and skin application ease was rated very well by the trial participants, they were able to spread it smoothly all over the skin. The participants were of the opinion that the cream is absorbed into the skin evenly and quickly (within 1 to 3 minutes) leaving a perceptible film and did not cause the feeling of stickiness or heaviness where applied.

Hydration

The cream significantly improves the skin hydration and does not provoke skin pulling sensation, but softens and smooth the skin, at the same time reducing the skin roughness, itching, skin irritation, redness, rosacea and squama. The skin became supple and more pleasant to the touch after the one application of the cream.

Photographic Evidence

Trial 1

The clinical results achieved in the following symptomatic patients are provided in detail below.

Photographic documentation supporting these therapeutic claims is provided in the FIGS. 1 to 4, illustrating the effect of applying the cream comprising a therapeutic amount of Kalanchoe pinnata according to Example 6, including in this instance: 9.6 g extract/juice of Kalanchoe pinnata; 7 g glycerol monostearate; 5 g cetyl alcohol; 34.4 g coconut oil; 43 g demineralized water; and 1 g phenoxyethanol.

FIG. 1

Patient: female aged 31

Condition/symptoms: atopic contact dermatitis on hands for 31 years

Results: 28 days of treatment (twice daily)

95% clearance of visible symptoms

FIG. 2

Patient: female aged 65

Condition/symptoms: psoriasis skin behind ear/scalp for 16 years

Results: 28 days of treatment (twice daily)

100% clearance of visible symptoms

FIG. 3

Patient: female aged 65

Condition/symptoms: psoriasis inner ear for 16 years

Results: 7 days of treatment (twice daily)

100% clearance of visible symptoms

FIG. 4

Patient: female aged 23

Condition/symptoms: allergic contact dermatitis on palm/hand for 11 years

Results: 7 days of treatment (twice daily) with product of example X

95% clearance of visible symptoms

Trial 2

Further photographic documentation supporting the therapeutic claims is provided in the FIGS. 5 to 11 which illustrate the effect of applying a cream comprising a therapeutic amount of both Kalanchoe pinnata and Kalanchoe daigremontiana, according to Example 6a. The formulation used for all the patients in this particular instance comprised 4.8 g extract/juice of Kalanchoe pinnata; 4.8 g of the extract/juice of Kalanchoe daigremontiana; 7 g glycerol monostearate; 5 g cetyl alcohol; 34.4 g coconut oil; 43 g demineralized water; 1 g phenoxyethanol.

FIG. 5

Patient: female aged 22

Condition/symptoms: acne back lesions

Results: 6 days of treatment (twice daily) with combination product

85% reduction in visible symptoms

FIGS. 6 and 7

Patient: female aged 41

Condition/symptoms: psoriasis on hands and arms for 3 years—large patch-like psoriatic plaques, clearly separated from the healthy skin, covered with squama.

Results: 21 days of treatment (twice daily) with combination product

100% clearance of visible symptoms

FIGS. 8 and 9

Patient: female aged 34

Condition/symptoms: psoriasis on scalp and leg for 17 years

Results: 19 days of treatment (twice daily) with combination product

100% clearance of visible symptoms

FIG. 10

Patient: female aged 23

Condition/symptoms: allergic contact dermatitis on fingers for 8 years, steroids previously unsuccessful.

Results: 2 days (twice daily)—with combination product

95% reduction in visible symptoms

FIG. 11

Patient: female aged 38

Condition/symptoms: atopic contact dermatitis fingers for 33 years, steroids previously unsuccessful.

Results: 10 days (twice daily) with combination product

95% reduction in visible symptoms

Trial 3

Further photographic documentation supporting the therapeutic claims is provided in the FIG. 12 which illustrates the effect of applying a cream comprising a therapeutic amount of Kalanchoe daigremontiana according to Example 1b for the specific treatment of acne, especially facial acne. The formulation comprised 9.6 g extract/juice of Kalanchoe Daigremontiana; 7 g glycerol monostearate; 5 g cetyl alcohol; 34.4 g coconut oil; 43 g demineralized water; and 1 g phenoxyethanol.

FIG. 12

Patient: female aged 22

Condition/symptoms: facial and neck acne lesions

Results: 6 days (twice daily) with single product

85% reduction in visible symptoms

In-Vitro Testing for Anti-Viral Effect

The treatment of viral infection, particularly the herpes HHV-1 virus, presents several challenges as viruses in this family develop in the host cell, rely on its metabolism to “automatically” affecting host cell activity but are not entirely eliminated systematically even after the clinical symptoms appear to be gone.

The most effective treatment for herpes would require specific action, low toxicity, no carcinogenic action and of favourable pharmacokinetic parameters. Presently one medication of choice in prophylaxis and prevention of HHV-1 infections is acyclovir. Acyclovir is a selective inhibitor of DNA replication and exhibits a degree of toxicity in relation to host cells. Adverse effects dictate that this treatment must not be used by pregnant and breastfeeding women. Other undesirable adverse effects include headaches and dizziness, nausea, vomiting, diarrhoea, stomach ache, pruritus, rash, hypersensitivity to light, tiredness and fever.

There is very little known about the anti-viral properties of many plant based extracts and therefore the applicants have designed and undertaken in vitro model testing of human keratinocyte cell cultures infected with herpes virus type 1 (HHV-1) in the presence of Kalanchoe daigremontiana and made a comparison with the toxicity and efficacy of the known treatment: Acyclovir

1. Cytotoxic Effect

Firstly, the cytotoxic effect of Kalanchoe daigremontiana extract on human keratinocyte cells (HaCaT) without infection was determined. HaCaT culture was incubated with various dilutions of the extract to determine its cytotoxic effects on skin cells. The ratio of extract to culture fluid was Extract:Culture Fluid (1:0; 1:2; 1:4; 1:9; 1:11). Cytotoxic activity of the tested solution in this MTT assay is the value of IC50 inhibitory concentration, i.e. a concentration with which the proliferation/viability of cells is inhibited by 50% in comparison with control.

After 24 hrs and 48 hrs a decrease in absorbance indicated lower cell viability in comparison with positive control cells—see Table 1, Table 2 and Table 3 (below).

TABLE 1
Absorbance values for MTT assay at 570 nm wavelength subtracted by the background
measured with 630 nm wavelength. Rejected outliers are underlined.

| | 1 | 2 | 3 | 4 | 5 | 6 | 7 | 8 | 9 | 10 | 11 | 12 |
|---|---|---|---|---|---|---|---|---|---|---|---|---|
| A | 0.743 | 0.41 | 0.263 | 0.604 | 0.791 | 0.692 | 0.907 | 0 | 0 | 0 | 0 | 0 |
| B | 0.521 | 0.376 | 0.749 | 0.717 | 0.421 | 0.37 | 0.224 | 0.571 | 0.243 | 0.672 | 0.215 | 0.472 |
| C | 0.21 | 0.111 | 0.166 | 0.201 | 0.235 | 0.092 | 0.093 | 0.187 | 0.157 | 0.206 | 0.228 | 0.117 |
| D | 0.165 | 0 | 0.016 | 0 | 0 | 0 | 0 | 0 | 0 | 0 | 0 | 0 |
| E | 0.363 | 0.385 | 0.324 | 0.588 | 0.47 | 0.413 | 0.477 | 0.489 | 0.665 | 0.55 | 0.59 | 0.637 |
| F | 0.426 | 0.219 | 0.188 | 0.279 | 0.273 | 0.258 | 0.233 | 0.266 | 0.318 | 0.351 | 0.262 | 0.151 |
| G | 0.042 | 0.004 | 0.001 | 0.002 | 0.004 | 0.003 | | | | | | |
| H | 0.038 | 0.038 | 0.044 | 0.04 | 0.038 | 0.046 | | | | | | |

TABLE 2
Mean absorbance values for MTT assay above. Corrected values are the
ones diminished by the absorbance for a blank test.

| | | | | | Standard |
|---|---|---|---|---|---|
| | | | Standard | Corrected | deviation |
| Ratio of extract: culture fluid | | Mean | deviation | mean | corrected |
| | Blank test | 0.003 | 0.001 | | |
| | Positive control | 0.648 | 0.150 | 0.645 | 0.149 |
| | Negative control | 0.000 | 0.000 | 0.000 | 0.000 |
| 24 hr | 1 to 0 | 0.481 | 0.145 | 0.478 | 0.144 |
| incubation | 1 to 2 | 0.185 | 0.048 | 0.182 | 0.047 |
| | 1 to 4 | 0.003 | 0.007 | 0.000 | 0.006 |
| | 1 to 9 | 0.391 | 0.055 | 0.388 | 0.054 |
| | 1 to 11 | 0.243 | 0.039 | 0.241 | 0.037 |
| 48 hr | 1 to 0 | 0.345 | 0.165 | 0.342 | 0.164 |
| incubation | 1 to 2 | 0.179 | 0.043 | 0.176 | 0.042 |
| | 1 to 4 | 0.000 | 0.000 | 0.000 | 0.000 |
| | 1 to 9 | 0.549 | 0.067 | 0.546 | 0.066 |
| | 1 to 11 | 0.286 | 0.048 | 0.283 | 0.046 |

TABLE 3
The percentage of live cells calculated in relation to positive
control, where the viability was assumed as 100%.

| | | Cell |
|---|---|---|
| Ratio extract: culture fluids | | viability |
| Positive control | | 100% |
| 24 h incubation | 1 to 0 | 74% |
| | 1 to 2 | 28% |
| | 1 to 4 | 0% |
| | 1 to 9 | 60% |
| | 1 to 11 | 37% |
| 48 h incubation | 1 to 0 | 53% |
| | 1 to 2 | 27% |
| | 1 to 4 | 0% |
| | 1 to 9 | 85% |
| | 1 to 11 | 44% |

Concentration corresponding to IC50 value is underlined. The IC50 for Kalanchoe daigremontiana extract was observed in the 1:9 extract after 24 hr incubation and 1:0 dilutions (undiluted extract) after 48 hr incubation. The lowest decrease of viability was noted for undiluted extract (1:0) and two higher dilutions: 1:9 and 1:11. Cell viability of HaCaT line determined after 24 hr and 48 hr incubations is also graphically shown in FIG. 13.

2. HaCaT Cell Viability

The cell viability for the HaCaT cell culture incubated over 24 hrs with Kalanchoe daigremontiana extract in respective dilutions is further illustrated in the graph of FIG. 14. Keratinocyte cell viability incubated with Kalanchoe daigremontiana was lower in comparison with the control. The viability appears generally higher in the greater dilutions and in the 1:11 extract dilution the viability decreased only by approximately 25% in comparison with control.

Next, cell viability additionally in the presence of the Herpes virus was investigated with a Cell Count Assay (NucleoCounter NC-3000). An HHV-1 suspension was added to HaCaT cell line and incubated for 1 hour to enable adsorption of virus to cells; the cells were then incubated with the extract, in the respective dilution ratios, for 24 hrs.

TABLE 5
Analysis of HaCaT cell line viability infected with HHV-1 and incubated
with Kalanchoe daigremontiana extract (in dilutions).

| | | All cells | | | Dead cells | | | |
|---|---|---|---|---|---|---|---|---|
| | | Number | | Mean | Number | | Mean | Viability |
| Name | | of cells | Cell % | intensity | of cells | Cell % | intensity | [%] |
| Ratio - | Control | 4225 | 92.39 | 211132.5 | 447 | 10.58 | 158128.9 | 89.4 |
| extract: | Control + HSV1 | 5176 | 88.49 | 196948.1 | 1099 | 21.23 | 176139.1 | 78.8 |
| culture | 1 to 0 | 263 | 19.4 | 37280 | 250 | 91.01 | 596593.6 | 4.9 |
| fluid | 1 to 4 | 1065 | 57.23 | 57088.3 | 911 | 95.06 | 591548.7 | 14.5 |
| | 1 to 9 | 3563 | 63.02 | 125352.5 | 1306 | 85.54 | 127733.5 | 63.3 |
| | 1 to 11 | 4827 | 73.33 | 119939.4 | 1161 | 36.65 | 145520.8 | 75.9 |
| | Control + acyclovir | 7648 | 79.67 | 160070.3 | 5683 | 24.05 | 152872.9 | 25.7 |

A graphic indicating the percentage of HaCaT line cell viability infected with HHV-1 and incubated with various dilutions of Kalanchoe daigremontiana extracts is also shown in FIG. 15.

The HaCaT line cells incubated with HHV-1 were characterised by about 10% lowered viability.

The addition of Kalanchoe daigremontiana extract to the infected cultures additionally lowered their viability (after 24 hr incubation) but with greater dilution of the extract, the cell viability was higher. The 1:11 extract dilution enabled the viability to remain almost the same level in comparison with positive control, whereas the 1:9 dilutions lowered the viability by about 15%.

It is noted that the comparison with Acyclovir, the reference medication which is typically administered in case of HHV-1 infection, lowered the cell viability considerably to 25%.

3. HaCaT Cell Quantitative Viability

Vitality VB-48 Assay (NucleoCounter NC-3000) was used to determine the percentage estimation of keratinocytes of varied degrees of viability after 24 h incubation with Kalanchoe daigremontiana extract.

The HaCaT cell culture incubated (24 h) with Kalanchoe daigremontiana extract of respective dilution was further investigated and the results provided in Table 7.

TABLE 7

| | | | | lower | |
|---|---|---|---|---|---|
| | | | dead | viability | healthy |
| Name | | | cells % | cells % | cells % |
| Ratio-extract: | Control | Mean | 12.85 | 5.13 | 81.99 |
| culture fluid | | Standard | 4.43 | 1.74 | 6.08 |
| | | deviation | | | |
| | 1 to 0 | Mean | 83.26 | 16.55 | 0 |
| | | Standard | 5.41 | 5.31 | 0 |
| | | deviation | | | |
| | 1 to 2 | Mean | 83.55 | 16.27 | 0.05 |
| | | Standard | 4.07 | 4.15 | 0.05 |
| | | deviation | | | |
| | 1 to 4 | Mean | 82.24 | 17.52 | 0.11 |
| | | Standard | 5.96 | 5.9 | 0.11 |
| | | deviation | | | |
| | 1 to 9 | Mean | 56.76 | 34.49 | 8.18 |
| | | Standard | 1.83 | 0.98 | 1.4 |
| | | deviation | | | |
| | 1 to 11 | Mean | 53.72 | 34.56 | 11.37 |
| | | Standard | 9.19 | 8.52 | 1.12 |
| | | deviation | | | |

The percentage estimation of keratinocytes that are dead, of lowered viability or healthy after 24 h incubation with Kalanchoe daigremontiana extract is further illustrated in FIG. 16.

An HHV-1 suspension was added to HaCaT cell line and incubated for 1 hour to enable adsorption of virus to cells. Afterwards, the cells were incubated with a plant extract of the respective dilution for 24 hrs to assess the viability of the keratinocytes with HHV-1 incubated with the respective dilutions of the extract of the invention as compared with Acyclovir.

| | | | | lower | |
|---|---|---|---|---|---|
| | | | dead | viability | healthy |
| Name | | | cells % | cells % | cells % |
| Ratio-extract: | Control | Mean | 12.85 | 5.13 | 81.99 |
| culture fluid | | Standard | 4.43 | 1.74 | 6.08 |
| | | deviation | | | |
| | Control + | Mean | 26.61 | 1.02 | 61.22 |
| | HSV1 | Standard | 7.17 | 2.1 | 9.04 |
| | | deviation | | | |
| | 1 to 0 | Mean | 93.84 | 6.13 | 0 |
| | | Standard | 3.25 | 3.25 | 0 |
| | | deviation | | | |
| | 1 to 4 | Mean | 69.08 | 29.56 | 0.83 |
| | | Standard | 1.47 | 1.49 | 1.36 |
| | | deviation | | | |
| | 1 to 9 | Mean | 49.41 | 34.85 | 15.47 |
| | | Standard | 4 | 5.4 | 1.41 |
| | | deviation | | | |
| | 1 to 11 | Mean | 47.56 | 23.68 | 28.47 |
| | | Standard | 2.52 | 2.58 | 1.18 |
| | | deviation | | | |
| | Control + | Mean | 77.1 | 19.51 | 3.27 |
| | acyclovir | Standard | 5.3 | 6.12 | 0.88 |
| | | deviation | | | |

FIG. 17 further illustrates the keratinocytes infected with HHV-1 that are dead, of lowered viability or healthy with a control and after 24 h incubation with Kalanchoe daigremontiana extract as compared with acyclovir.

After keratinocytes infection with HHV-1 the number of viable cells decreased by 20%. It is noted that the viability of keratinocytes incubated for 24 hrs using Kalanchoe daigremontiana extract was decreased. With the greater dilution of the extract, the percentage of dead cells decreases and the percentage of lower viability and healthy cells increases. After diluting the extract to 1:9 or 1:11 about 50% cells were viable or of lowered viability and about 50% were dead. Application of acyclovir in the culture resulted in only 25% of live/lower viability cells. Proliferation of keratinocytes incubated using Kalanchoe daigremontiana extract was inhibited. Incubation of HHV-1 infected cells with the extract was found to additionally lower cell proliferation. However, the more diluted extract was used the higher number of active and lower-activity cells were present. The number of HaCaT cells in S and G2/M phase increases, which may indicate that they proliferate more intensively than the control cells.

Keratinocytes incubated with Kalanchoe daigremontiana extract for 24 hours show mitochondrial membrane potential drop. The use of Kalanchoe daigremontiana extract at volume ratios of 1:9 and 1:11 results in the slightest mitochondrial membrane potential drop in the studied samples and 1:9 volume ratio appears to be the most desirable for mitochondrial membrane potential. Incubation with the extract leads to the decrease of mitochondrial membrane potential of HaCaT cells, previously infected with HHV-1.

4. HaCaT Cell Morphology

Confocal microscopy (microscope magnification—60×, digital zoom) was used to view the cell structure status and positioning 48 hrs after infection with HHV-1 (FIG. 18).

The method of direct immunofluorescence staining wherein the nucleus—blue (A), viral antigen —green (B), actin—red (C), nucleus/actin (D) helps further understand the structural changes in the cells.

Next FIG. 19 shows that when acyclovir is added to the HaCaT cell line infected with HHV-1, a lower amount of viral antigen is found, which indicates lower virus replication. The apoptotic (dead) cells of condensed chromatin are clearly visible.

The same confocal microscopy technique was used to view the cell structure status and positioning after HHV-1 infection and treatment with Kalanchoe daigremontiana. As before the method of direct immunofluorescence staining is nucleus—blue (A), viral antigen—green (B), actin—red (C), nucleus/actin (D).

FIG. 20 shows the HaCaT cell line infected with HHV-1 and treated with extract of the invention 1:9 (dilution ratio) at 24 hrs; and FIG. 21 shows the same cells at 48 hrs.

FIG. 22 shows the HaCaT cell line infected with HHV-1 and treated with extract of the invention 1:11 (dilution ratio) at 24 hrs; and FIG. 23 shows the same cells at 48 hrs.

The culture of keratinocytes infected with HHV-1 incubated with the extract of the invention, in FIGS. 22 and 23, showed a large number of cells of irregular shape and varied sizes and characteristic symptoms of cytopathic effects (CPE)—syncytial effect.

Additionally, incubation with the extract led to the development of a large number of cells which were visually symptomatic of the apoptotic process. An apoptotic cell rounds, the nuclear chromatin condenses and cytoskeleton disintegrates (e.g. FIGS. 21, 22 and 23) suggesting initiation of program cell death.

These changes were also accompanied by a decrease of the number of cells containing viral antigens, in comparison with the culture in FIG. 18 which was not incubated with the extract of the invention.

During apoptosis, proteins and nucleic acids both of a cell as well as of an infecting virus degrade. Death of an infected cell therefore prevents the unwanted infection from spreading. It is important in the viral mechanism is to counteract the cells natural destruction method, in case of HHV-1 the mechanism of survival depends on the ability to establish latent infection and the viral ability to hinder an infected cell death by inhibiting the apoptotic pathway.

Occurrence of keratinocytes apoptosis after incubation with plant extract therefore appears to have a positive impact since—it leads to the elimination of HHV-1 infected cells and thus hinders the latent spread of viruses to healthy cells.

Following incubation with the extract the ratios of individual cell cycle phases of human keratinocytes previously infected with HHV-1 are changed. Lower extract dilutions make the majority of cells move to G1 sub-phase, in which DNA contents are the lowest (apoptosis is likely to occur). With greater extract dilutions the number of cells in G1 sub-phase decreases and the number of cells in G1/G0 phase (growth phase) increases. The number of cells in S and G2/M phases is similar.

5. DNA Analysis

Analysis of viral DNA in keratinocytes with real-time PCR was performed.

The graphic chart in FIG. 24 displays the results of Real-time PCR analysis of HaCaT cells (at 24 hrs and 48 hrs after infection) with HHV-1 and incubated with Kalanchoe daigremontiana plant extract 1 hr after HHV-1 adsorption. The negative control (K−) was non-infected keratinocytes and positive control (K+) was HHV-1 infected cells. Also, to provide a direct comparison with the extract and its medical use of the invention, an additional incubation with acyclovir was used where Acyclovir was added 1 hr after HHV-1 adsorption.

The graphic chart in FIG. 25 displays the results of Real-time PCR analysis of HaCaT cells (at 24 hrs and 48 hrs after infection) with HHV-1 and incubated with Kalanchoe daigremontiana plant extract with no 1 hr pre-adsorption (before culture infection). The negative control (K−) was non-infected keratinocytes and positive control (K+) was HHV-1 infected cells, as before. Also, to provide a direct comparison with the extract and its medical use of the invention, an additional incubation with acyclovir was used but added before culture infection.

The results showed a decrease in the number of DNA viral copies following the administration of Kalanchoe daigremontiana extract as compared to the samples incubated with acyclovir.

These tests further indicated a greater inhibition of HHV-1 replication when the extract was given prior to HHV-1 culture infection (FIG. 25) even to DNA copy numbers not detected with Real-time method (PCR). The applicants therefore consider it probable that Kalanchoe daigremontiana extract provided in accordance with a composition of the invention could hinder the process of adsorption of the virus HHV-1 on the surface of skin cells.

In summary, the compositions of the invention as disclosed in the present application have been shown to be effective to combat several dermal conditions, including medical conditions such as dermatitis, dermal acne and dermal herpes. The invention further treats the irritations such as itching, symptoms of roughness, exfoliation and the pulling and burning sensation associated with those dermal conditions.

The composition is effective in speeding up regeneration and recovery of the natural protective layer of the skin and therefore is particularly effective for the before described medical conditions.

Various further aspects and embodiments of the present invention will be apparent to those skilled in the art in view of the present disclosure.

All documents mentioned in this specification are incorporated herein by reference in their entirety. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described. Various further aspects and embodiments of the present invention will be apparent to those skilled in the art in view of the present disclosure. While the invention has been described herein in reference to specific aspects, features and illustrative embodiments of the invention, it will be appreciated that the utility of the invention is not thus limited, but rather extends to and encompasses variations, modifications and alternative embodiments, as will be understood by those of ordinary skill in the field.

1. (canceled)

2. A topical composition comprising:
a therapeutically effective amount of Kalanchoe daigremontiana extract,
wherein the amount of Kalanchoe daigremontiana is therapeutically effective for the treatment or prevention of at least one dermal condition, and
wherein the composition is formulated as a cream, foam, gel, lotion, ointment, or shampoo.

3. The topical composition of claim 2 wherein the dermal condition is HHV-1 infection.

4. The topical composition of claim 3 wherein the therapeutically effective amount of Kalanchoe daigremontiana extract is in the range of 0.01% to 30% w/w.

5. The topical composition of claim 4, wherein the topical composition further comprises at least one pharmaceutically or cosmetically acceptable excipient.

6-7. (canceled)

8. The composition of claim 5 wherein the composition comprises 35-75% w/w demineralized water and/or 4-20% w/w glycerol monostearate.

9. The composition of claim 5 wherein the excipient or excipients includes a mixture of fats comprising glycerol monostearate and one or more of beeswax, coconut oil, cetyl alcohol, shea butter, and Cannabis sativa (Hemp) Seed Oil.

10. The composition of claim 9, wherein the composition comprises 4-28% w/w of beeswax, 4-40% w/w of coconut oil, 2-25% w/w of cetyl alcohol, and/or 2-10% w/w of shea butter.

11. The composition of claim 10, wherein the composition further comprises allantoin.

12. The composition of claim 11 wherein the composition further comprises 0.5 to 1% w/w phenoxyethanol.

13-25. (canceled)

26. The topical composition of claim 2 further comprising a therapeutically effective amount of Kalanchoe pinnata extract wherein the amount of Kalanchoe pinnata is therapeutically effective for the treatment or prevention of at least one dermal condition, wherein the dermal condition is selected from the group consisting of atopic dermatitis, allergic contact dermatitis, psoriasis, and acne.

27. The topical composition of claim 26 wherein the therapeutically effective amount of Kalanchoe pinnata extract is in the range of 0.01% to 30% w/w.

28. The topical composition of claim 26 wherein the topical composition further comprises at least one pharmaceutically or cosmetically acceptable excipient.

29. The composition of claim 28 wherein the composition further comprises 35-75% w/w demineralized water and/or 4-20% w/w glycerol monostearate.

30. The composition of claim 28 wherein the excipient or excipients includes a mixture of fats comprising glycerol monostearate and one or more of beeswax, coconut oil, cetyl alcohol, shea butter, and Cannabis sativa (Hemp) Seed Oil.

31. The composition of claim 30, wherein the composition comprises 4-28% w/w of beeswax, 4-40% w/w of coconut oil, 2-25% w/w of cetyl alcohol, and/or 2-10% w/w of shea butter.

32. The composition of claim 30, wherein the composition further comprises allantoin.

33. The composition of claim 32 wherein the composition further comprises 0.5 to 1% w/w phenoxyethanol.

34. A method of skin care or treating a dermal condition, the method comprising:
applying a composition for topical administration comprising a therapeutically effective amount of Kalanchoe daigremontiana extract or juice to an affected area.

35. The method of skin care or treating a dermal condition of claim 34 wherein the composition for topical administration further comprises at least one excipient for use in the treatment or prevention of a dermal condition.

36. The method of skin care or treating a dermal condition of claim 35 wherein the dermal condition is HHV-1 infection.

37. The method of skin care or treating a dermal condition of claim 36 wherein the composition for topical administration comprises 0.01% to 30% w/w of Kalanchoe daigremontiana extract or juice.

38. The method of skin care or treating a dermal condition of claim 37 wherein the composition for topical administration comprises 4-20% w/w glycerol monostearate and/or 35-75% w/w demineralized water.

39. The method of skin care or treating a dermal condition of claim 38 wherein the composition for topical administration further comprises one or more of beeswax, coconut oil, cetyl alcohol, shea butter, and Cannabis sativa (Hemp) Seed Oil.

40. A method of skin care or treating a dermal condition, the method comprising:
applying a composition for topical administration comprising a therapeutically effective amount of Kalanchoe daigremontiana and a therapeutically effective amount of Kalanchoe pinnata extract or juice to an affected area.

41. The method of skin care or treating a dermal condition of claim 40 wherein the composition for topical administration further comprises at least one excipient for use in the treatment or prevention of a dermal condition.

42. The method of skin care or treating a dermal condition of claim 41 wherein the dermal condition is selected from the group consisting of atopic dermatitis, allergic contact dermatitis, psoriasis, and acne.

43. The method of skin care or treating a dermal condition of claim 42 wherein the composition for topical administration comprises 0.01% to 30% of Kalanchoe daigremontiana extract or juice and 0.1-30% w/w of Kalanchoe pinnata extract or juice.

44. The method of skin care or treating a dermal condition of claim 43 wherein the composition for topical administration comprises 4-20% w/w glycerol monostearate and/or 35-75% w/w demineralized water.

45. The method of skin care or treating a dermal condition of claim 44 wherein the composition for topical administration further comprises one or more of beeswax, coconut oil, cetyl alcohol, shea butter, and Cannabis sativa (Hemp) Seed Oil.
